(19) United States
(12) Patent Application Publication
(10) Pub. No.: US 2016/0089399 A1
(43) Pub. Date: Mar. 31, 2016

(54) NON-HEMOLYZING BLOOD FILTER AND METHODS FOR FILTERING BLOOD WITHOUT HEMOLYSIS

(71) Applicants: Alan PERLSTEIN, Brooklyn, NY (US); Ashlesha MULEY, Brooklyn, NY (US); Xi HUANG, Montvale, NJ (US)

(72) Inventors: Alan PERLSTEIN, Brooklyn, NY (US); Ashlesha MULEY, Brooklyn, NY (US); Xi HUANG, Montvale, NJ (US)

(21) Appl. No.: 14/787,214

(22) PCT Filed: Apr. 25, 2014
(86) PCT No.: PCT/US2014/035576

§ 371 Date: Oct. 26, 2015

Related U.S. Application Data
(60) Provisional application No. 61/816,061, filed on Apr. 25, 2013.

(51) Int. Cl. A61K 35/18; B01D 15/36; A61M 1/34; A61M 1/36; C12N 5/078; G01N 21/79

(52) U.S. Cl. CPC A61K 35/18; C12N 5/0641; G01N 21/79; A61M 1/34; A61M 1/3679; B01D 15/36; A61K 2035/124

(57) ABSTRACT

An article, system, and method is provided for the filtration of blood wherein the blood is removed, contacted with a filter substrate operatively associated with a filter structure, and the filtered blood is subsequently returned to a receiver. Methods for removing iron from the liquid fraction of blood and for determining whether a substrate is capable of selectively retaining 2,2′-dipyrydyl (DP)-Fe²⁺ complexes are also disclosed.

### CROSS-REFERENCE TO RELATED APPLICATIONS

This application claims the benefit of U.S. Provisional Patent Application No. 61/816,061, filed Apr. 25, 2013, the entire contents of which are hereby incorporated by reference.

### BACKGROUND OF THE DISCLOSURE

Red blood cell (“RBC”) units are routinely stored for up to forty-two days. During RBC processing and storage, approximately 1-2% of RBCs undergo hemolysis ex vivo, releasing toxic oxidative components (“TOCs”), such as hemoglobin (“Hb”), heme, iron, and microparticles (“MPs”), which accumulate in the storage media. This process is illustrated in FIG. 1. Furthermore, approximately twenty-five percent of RBCs become damaged and are rapidly cleared post-transfusion, mostly within about two hours. The lysed RBCs and TOCs collectively called the “storage lesion,” can overwhelm the subject's ability to clear these transfusion byproducts, resulting in enhanced complications and adverse outcomes. Hess, J. R., “Measures of stored red blood cell quality,” Vox Sang. (Jan. 22, 2014) [Epub ahead of print]; Cohen, B., and Matot, I., “Aged erythrocytes: a fine wine or sour grapes?” Br. J. Anaesth. 111(Suppl. 1):i62-70 (2013); Koch, C. G., et al., “Red blood cell storage: how long is too long?” Ann. Thorac. Surg. 96(5):1894-9 (2013); Hod, E. A., and Spitalnik, S. L., “Stored red blood cell transfusions: Iron, inflammation, immunity, and infection,” Transfus. Clin. Biol. 19(3):84-9 (2012). These enhanced complications and adverse outcomes include increases in sepsis, pneumonia, organ failure, myocardial infarction, thrombosis, and mortality. Isbister, J. P., et al., “Adverse blood transfusion outcomes: establishing causation,” Transfus. Med. Rev. 25(2):89-101 (2011); Triulzi, D. J., and Yazer, M. H., “Clinical studies of the effect of blood storage on patient outcomes,” Transfus. Apher. Sci. 43(1):95-106 (2010); van de Watering, L. M., and Brand, A., “Effects of storage of red cells,” Transfus. Med. Hemother. 35(5):359-67 (2008); Seghatchian, J., and de Sousa, G., “An overview of unresolved inherent problems associated with red cell transfusion . . . ,” Transfus. Apher. Sci. 37(3):251-9 (2007). To limit the risks involved, practitioners often avoid providing RBC transfusions. See Slight, R. D., et al., “Red cell transfusion in elective cardiac surgery patients: where do we go from here?” Br. J. Anaesth. 102 (3) :294-6 (2009) ; and Madjdpour, C., and Spahn, D. R., “Allogeneic red blood cell transfusions: efficacy, risks, alternatives and indications,” Br. J. Anaesth. 95(1):33-42 (2005). If RBC transfusions were made safer, this shift in decision-making would not occur and the patients' quality of life would improve.

In the United States, approximately fifteen million packed RBC units are collected and stored, and these units are transfused to approximately five million patients yearly, thereby making RBC transfusion one of the most commonly performed medical procedures. Until now, there has been no solution to mitigate the risks resulting from the RBC storage lesion.

The mammalian body has no active mechanism for the excretion of excess iron. Iron homeostasis thus relies on the amount of iron that is absorbed from the small intestine versus the amount of iron lost by passive processes. During normal physiology, the amount of iron absorbed (1-2 mg/day) is offset by an amount of iron lost by sloughing of intestinal mucosa and skin, as well as small amounts lost in the urine and bile. Andrews, N. C., “Disorders of iron metabolism,” N. Engl. J. Med. 341(26): 1986-95 (1999) and erratum at N. Engl. J. Med. 342(5):364 (2000). As iron is needed by virtually all body cells and especially erythrocytes, the mammalian body's day-to-day iron requirements are met by recycling between various compartments.

Transfusion of blood is, of course, well known in the art. Each unit of transfused red blood cells has approximately 200-250 mg of “free” iron, i.e., iron not encapsulated within erythrocytes. Because the mammalian body has no active mechanism for the excretion of excess iron, free iron gets deposited in the organs. And, because free iron has high oxidative potential and is capable of oxidizing cell membranes, proteins, and DNA, the free iron damages the cells, tissues, and eventually organs where it is deposited.

Some subjects, noticeably those with thalassemia major, sickle cell disease, myelodysplastic syndrome, aplastic anemia, hemolytic anemia, and refractory sideroblastic anemias, may become transfusion-dependent. As a result of this dependency, these subjects receive large quantities of free iron that gradually accumulates in various tissues, causing morbidity and mortality. This accumulation is known as “iron overload.”

On a macro scale, subjects suffering from iron overload suffer from critical organ damage that includes the heart, brain, liver, and kidney, among others. Additional research has identified a higher association with obesity, diabetes, growth abnormalities, cancer, and average lower life span among these subjects. In subjects with myelodysplastic syndromes, this free iron is thought to be one of the major causes of progression of the disease to cancer due to its DNA-damaging nature.

The current approach for tackling iron overload involves use of FDA-approved chelation drugs that are administered intravenously or orally. These drugs, such as deferoxamine, defferiprone, and defafisirox, are thought to bind free iron in the tissues; the kidneys excrete the chelation drug-iron complex. While these chelation drugs are effective in managing iron homeostasis in cases of iron overload, these drugs do not address the underlying cause of iron overload. In addition, these treatments require long hospitalizations, have undesirable side effects, and have poor compliance rates among younger subjects.

Transfusion-based iron overload is a serious problem affecting millions of people all over the world. Current therapies only treat the condition once it occurs. The only blood filtration method currently approved involves passing processed blood through a leuko-reduction filter. This filter has its own secondary blood storage container, typically, a blood storage bag.

Thus, there is a long-felt but unmet need for the removal of free iron from blood products prior to transfusion. Of course, novelty and improvement is desirable in this, as in any, art.

### SUMMARY OF THE DISCLOSURE

Disclosed is an in-line addition to current processing system eliminating the need for secondary storage containers. However, secondary storage containers can also be used.

“Filtration” according to the present disclosure includes gravitational, mechanical/peristaltic, microfluidic and combinations thereof. During mechanical pumping, the conditions are optimized to avoid any negative effects on the blood and all blood components, such as sheer on red blood cells.

Disclosed is a method for removing free iron, free heme, free hemoglobin, damaged red blood cells and combinations thereof from the transfused blood.

Without being tied to any particular theory or result, it is contemplated that the disclosed in-line addition configuration will increase the overall efficiency of the blood transfusion, enhance the quality of blood, and reduce the clearance of transfused blood in patients.

Again, without being tied to any particular theory or result, it is also contemplated that the disclosed inline addition be used for veterinary purposes, for removal of aforementioned damaging components through kidney dialysis using same or modified principle.

Disclosed is a system for filtering blood comprising at least one substrate for chemical filtration of blood and blood components; and at least one structure for supporting; said substrate, wherein said structure is constructed and arranged with a porosity of between 0.0001 micron-20 micron. The substrate can be at least one of: a chelating agent; a styrene-divinylbenzene co-polymer containing iminodiacetic acid groups; polyphenols; phytates; ascorbic acid derivatives; polymeric hydroxamic acid; derivatives/hydrogels/resins, or chemical modifications of existing oral chelating drugs—deferoxamine, defferiprone, and defarisirox; or combinations thereof. The substrate can also be positioned in-line in a blood transfusion system. The substrate can also be positioned in-line in a blood transfusion system and blood is caused to make contact with said substrate by gravitational movement of blood, mechanical movement of blood, or combinations thereof.

Also disclosed is a method for removing iron from the liquid fraction of blood comprising contacting the liquid fraction of blood with a styrene-divinylbenzene co-polymer comprising iminodiacetic acid groups. The blood can be mammalian blood. The mammal can be selected from the group consisting of a human, a monkey, a chimpanzee, a dog, a cat, a rat, and a mouse. The mammal can be a human. The liquid fraction of blood can flow with respect to the styrene-divinylbenzene co-polymer comprising iminodiacetic acid groups. The flow can result from gravitational motion. The flow can also result from mechanical motion of blood.

Further disclosed is a method for determining whether a substrate is capable of selectively retaining 2,2′-dipyrydyl (DP)-Fe²⁺ complexes, comprising containing the substrate with a solution of DP-Fe²⁺ and determining the amount of DP-Fe²⁺ retained by the substrate, wherein the substrate does not significantly retain Arsenazo III-Ca²⁺ complexes. The ratio of the amount of retained DP-Fe²⁺ complexes to that of Arsenazo III-Ca²⁺ complexes can be 10:1. The ratio of the amount of retained DP-Fe²⁺ complexes to that of Arsenazo III-Ca²⁺ complexes can be 100:1. The ratio of the amount of retained DP-Fe²⁺ complexes to that of Arsenazo III-Ca²⁺ complexes can be 1,000:1. The ratio of the amount of retained DP-Fe²⁺ complexes to that of Arsenazo III-Ca²⁺ complexes can be greater than 1,000:1.

### BRIEF DESCRIPTIONS OF THE DRAWINGS

FIG. 1 illustrates proposed storage lesions that red blood cells can undergo with time, and is reprinted from Buehler, P. W., et al., “Blood aging, safety, and transfusion: capturing the ‘radical’ menace,” Antioxid. Redox Signal. 14(9):1713-28 (2011).

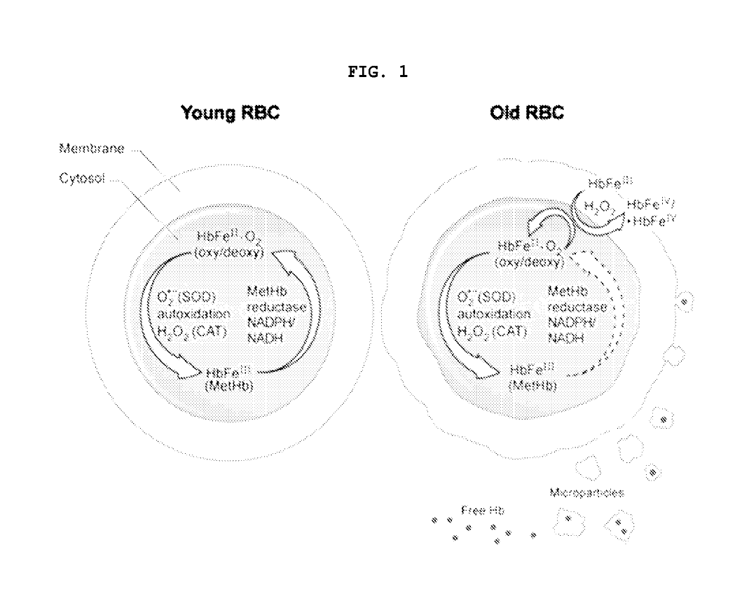

FIG. 2 demonstrates that Chelex® 100 resin retains DP-Fe²⁺.

FIG. 3 demonstrates that Chelex® 100 resin efficiently retains 1 mM DP-Fe²⁺, but not 1 mM Arsenazo III-Ca²⁺.

### DETAILED DESCRIPTION OF THE PREFERRED EMBODIMENT

Disclosed is an article, system, and method that decreases the overall burden of iron overload and other damaging components in blood transfusions. Without being tied to any particular theory or result, it is believed that binding and separating the dangerous components that trigger iron overload by removing them once they come into contact with the active agents of the device is disclosed.

Based on a long-felt but unmet need to remove dangerous components, disclosed is a chelation-based blood filter that will function in removing free or suspended dangerous elements and compounds before it enters into patient body and causes harm to the vital organs and other complications. This filter is capable of being included in the blood transfusion process at two stages: after the processing of blood in the blood collection centers and at the end stage just before the blood is transfused to the subject. Disclosed is a non-hemolysing filter that will remove (i) damaging components; and (ii) damaged red blood cells.

Disclosed is a filter that can be round/spherical in shape, integrated or separated into a single or multiple compartments, in series to the outlet of blood bag going to patients/inserted in blood bag during storage, made of biocompatible/new material, using chelating chemicals, immobilized/suspended, sandwiched/directly coming in contact with blood, where blood will flow over or flow through the filter and damaging components will be entrapped in the filter.

Without being tied to any particular theory or result, any of the following components in any combinations are contemplated being used in the disclosed article: alloys, ceramics, cellulose, plastic/polymers, thermoplastics, thermosets, elastomers, homopolymers, copolymers, polymer blends, polyvinylchloride, polyethelene, polypropylene, cycloolefin polymers/copolymers, polystyrene, acrylics, polycarbonates, polyurethanes, polyacetals, polyesters, polylactic acid copolyesters, polyanddes, polysulfones, polyimides, polyamide-imides, polyphenylene sulfide, polyether ether ketones, liquid crystalline polymers, nitrocellulose, fibrin, nanoplastics, nylon, nanoparticles, fluoropolymers styrenics, silicones, and biopolymers. These components can be coated, non-coated, or both, hydrophobic, hydrophilic, or both, and these plastics can be used in any combination with each other. Additionally, any of these components can be mixed with nano additives, plastic additives, or drugs.

Disclosed are custom membranes including, but not limited to: alloys, ceramics, mixed cellulose ester, polyesteramide, polyfluortetraethylene, polyethersolphone, polyvinylidene fluoride, polypropylene, cellulose acetate, glass fiber, quartz fiber polystyrene, polyether urethane, sulfated polyethylene, hydroxyathyl methacrylate, polylactic acid polyethylene glycol polycarbonate. These custom membranes may be coated and non-coated, hydrophillic, hydrophobic, or dual sided, or come in one or more layers. These materials can be used in any combination with each other.

Also disclosed is one or more chemical chelation components. Further disclosed is Chelex®, which is a chelating material from Bio-Rad Laboratories, Inc. capable of purifying other compounds via ion exchange. It is noteworthy for its ability to bind transition metal ions. Chelex® is a styrene-divinylbenzene co-polymer containing iminodiacetic acid groups. Other disclosed chemicals include polyphenols, phytates, ascorbic acid derivatives, polymeric hydroxamic acid derivatives/hydrogels/resins, or even chemical modifications of already existing oral chelating drugs: deferoxamine, defferiprone, and defarisirox.

Any chemical, compound, substrate, or combinations thereof are operatively associated with the disclosed filter. The association includes any type of fixation, imbedding or attachment of the substrate to the filter in a manner such that the chemical components are in direct contact with blood being filtered in the present invention.

Disclosed is chemical immobilization. Additional disclosures include, but are not limited to and/or physical adsorption, entrapment, absorption, immobilization on beads/resin, and integration with plastics, membranes, biocompatible polymers, in existing tubing, or combinations thereof.

Also disclosed is a construction and arrangement that removes dangerous elements form the blood without leaching anything or changing the pH/chemistry or morphology of the blood cells. The chemical is immobilized with a unique filter that optimizes the blood flow through rate in which dangerous components from the blood are extracted. The configurations of the filter range from circular to oval to rectangular to square to and any shape that has 3 or more sides. Furthermore, the porosity range of the filter membrane can be from 0.0001 microns to 20 microns.

Additionally disclosed is a configuration in order to filter the red blood cells on the basis of size whereby red blood cells of particular sizes are removed.

Substances for separation are, of course, well known in the art. Some substances separate on the basis of ion exchange, in which ions of one charge are retained on the substances. An example of an ion exchange substance is Chelex®, which is a styrene-divinylbenzene co-polymer containing iminodiacetic acid groups. Chelex® is well known in the art as a substance for separation based on ion exchange, but disclosed herein for the first time is that Chelex® is capable of selectively retaining DP-iron complexes. See “Chelex® 100 and Chelex 20 Chelating Ion Exchange Resin Instruction Manual,” Bio-Rad Laboratories, Hercules, Calif. (LIT200 Rev B). Also disclosed for the first time is that Chelex® does not bind protein-calcium ion complexes, thereby establishing selectivity of protein-metal ion binding.

Containers for these substances for separation typically have a single lumen, but the diameter of the lumen of one end of the container is typically smaller than the diameter of the lumen of the other end of the container. In other words, these containers are/have at least one tapering section. Other typical containers are columns, but these columns are not sized for blood. The containers disclosed herein are not tapered and are sized for blood.

As used herein, the term “free iron” refers to iron not encapsulated within intact erythrocytes. Free iron can include Fe³⁺ ions, Fe²⁺ ions, hemoglobin, heme, and 2,2′-dipyrydyl-Fe²⁺. As also used herein, the term “drug-chelated iron” refers to complexes of deferoxamine, defferiprone, and defarisirox with iron ions.

### EXAMPLES

Some non-limiting examples follow.

### Example 1

In this example, the ability of Chelex® 100 resin to retain iron ions, iron complexes, and an iron complex in the presence of calcium ions was examined. Approximately 0.5 g of Chelex® 100 resin (149 μm particle size; Bio-Rad Laboratories, Inc., Hercules, Calif.) was packed in a filter tube with a 40 μm pore size bed. Solutions of 1 mM FeCl₂, 1 mM FeCl₂, 1 mM 2,2′-dipyrydyl (DP)-Fe²⁺, and 1 mM deferoxamine (DFO)-Fe³⁺ were prepared in water (Sigma-Aldrich, St. Louis, Mo.). A solution of 1 mM DP-Fe²⁺ and 10 mM CaCl₂ (Sigma-Aldrich, St. Louis, Mo.) in water was also prepared.

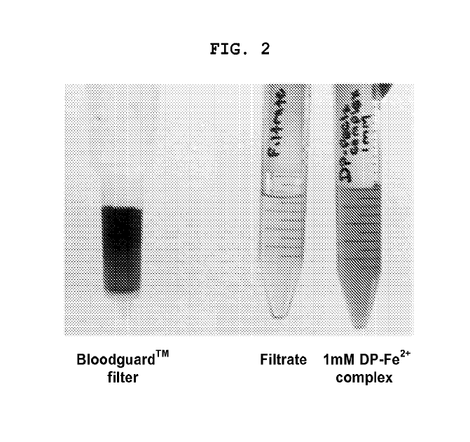

FIG. 2 demonstrates that Chelex® 100 resin retains DP-Fe²⁺. The right-hand tube in FIG. 2 is a 1 mM solution of DP-Fe²⁺. This solution was filtered through the Chelex® 100 resin filter tube at a rate of 4 ml/min and a contact time of fifteen seconds. The column on the far left of FIG. 2 demonstrates that the Chelex® 100 resin retains DP-Fe²⁺. After passing 93 ml of a 1 mM solution of DP-Fe²⁺ through 0.72 g of Chelex 100 resin, the filtrate began showing a slight pink color, indicating saturation (left-hand tube).

The capacities of Chelex® 100 resin to remove free Fe³⁺, Fe²⁺, DP-Fe²⁺, and DFO-Fe³⁺ complexes were titrated by adding measured amounts of solutions of the complexes until color appeared in the filtrates. The capacities of Chelex® 100 resin to retain each component are expressed below:

| | Amount of iron preparation retained |
|---|---|
| Preparation | (mg/g Chelex ® 100 resin) |
| Fe³⁺ | 103.7 |
| Fe²⁺ | 69.1 |
| DFO-Fe³⁺ | 10.1 |
| 1 mM DP-Fe²⁺ | 7.4 |
| 1 mM DP-Fe²⁺ | 4.8 |
| and 10 mM CaCl₂ | |

Thus, Chelex® 100 resin captures and retains iron even in the presence of strong iron chelators.

It is calculated that one gram of Chelex® 100 resin is sufficient to remove all hemoglobin-derived iron compounds in one unit of packed red blood cells. One unit of packed red blood cells contains approximately four-hundred milliliters of blood with hemoglobin levels at 150 grams per liter. Therefore, one unit of packed red blood cells contains approximately sixty grams of hemoglobin (150 g/L×0.4 L=60 g). Because the molecular weight of hemoglobin is approximately 64,500 daltons, one unit of packed red blood cells contains approximately 0.93 millimoles of hemoglobin (60 g÷64,500 Da≈0.93 millimoles). And, one molecule of hemoglobin contains four iron ions, each with a molecular weight of approximately fifty-six daltons. Therefore, there is approximately two-hundred and eight milligrams of iron in one unit of packed red blood cells (0.93 millimoles×4 iron ions×fifty-six daltons≈208 mg iron ions). Assuming a two percent hemolysis rate in any given unit of packed red blood cells, the amount of free iron in the packed red blood cell unit is approximately 4.2 milligrams. (208 mg iron ions×2%=4.2 mg iron ions). Because Chelex® 100 resin was found to retain at least 4.8 milligrams of iron ions per gram, one gram of Chelex® 100 resin is calculated to be sufficient to remove all hemoglobin-derived iron compounds in one unit of packed red blood cells.

### Example 2

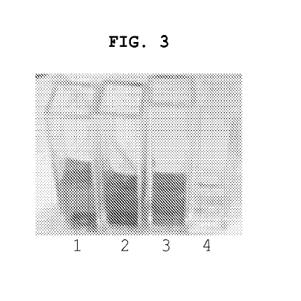

In this example, the relative selectivity of Chelex® 100 resin to retain iron complexes or calcium complexes was examined. Preparations of 2 mM DP-Fe²⁺ and 2 mM Arsenazo III-Ca²⁺ (Sigma-Aldrich, St. Louis, Mo.) in water were mixed in equal parts. FIG. 3 demonstrates that Chelex® 100 resin efficiently retains 1 mM DP-Fe²⁺, but not 1 mM Arsenazo III-Ca²⁺. Specifically, cuvette 2 contains the mixture of 1 mM DP-Fe²⁺ and 1 mM Arsenazo III-Ca²⁺ before the mixture was filtered through Chelex® 100 resin. Cuvette 1 contains the filtrate; note that the filtrate has the approximate color of a 1 mM Arsenazo III-Ca²⁺ solution in water. Cuvette 3 contains a 1 mM DP-Fe²⁺ solution in water before the mixture was filtered through Chelex® 100 resin. Cuvette 4 contains the filtrate; note that the filtrate is approximately colorless.

While the invention has been described in its preferred form or embodiment with some degree of particularity, it is understood that this description has been given only by way of example and that numerous changes in the details of construction, fabrication, and use, including the combination and arrangement of parts, may be made without departing from the spirit and scope of the invention.

What is claimed is:

1. A system for filtering blood comprising:
at least one substrate for chemical filtration of blood and blood components; and
at least one structure for supporting; said substrate, wherein said structure is constructed and arranged with a porosity of between 0.0001 micron-20 micron.

2. The system of claim 1, wherein said substrate is at least one of: a chelating agent; a styrene-divinylbenzene co-polymer containing iminodiacetic acid groups; polyphenols; phytates; ascorbic acid derivatives; polymeric hydroxamic acid; derivatives/hydrogels/resins, or chemical modifications of existing oral chelating drugs—deferoxamine, defferiprone, and defarisirox; or combinations thereof.

3. The system of claim 1 wherein said substrate is positioned in-line in a blood transfusion system.

4. The system of claim 1 wherein said substrate is positioned in-line in a blood transfusion system and blood is caused to make contact with said substrate by gravitational movement of blood, mechanical movement of blood, or combinations thereof.

5. A method for removing iron from the liquid fraction of blood comprising contacting the liquid fraction of blood with a styrene-divinylbenzene co-polymer comprising iminodiacetic acid groups.

6. The method of claim 5, wherein the blood is mammalian blood.

7. The method of claim 6, wherein the mammal is selected from the group consisting of a human, a monkey, a chimpanzee, a dog, a cat, a rat, and a mouse.

8. The method of claim 7, wherein the mammal is a human.

9. The method of claim 5, wherein the liquid fraction of blood is flowing with respect to the styrene-divinylbenzene co-polymer comprising iminodiacetic acid groups.

10. The method of claim 9, wherein the flow results from gravitational motion.

11. The method of claim 10, wherein the flow results from mechanical motion of blood.

12. A method for determining whether a substrate is capable of selectively retaining 2,2′-dipyrydyl (DP)-Fe²⁺ complexes, comprising containing the substrate with a solution of DP-Fe²⁺ and determining the amount of DP-Fe²⁺ retained by the substrate, wherein the substrate does not significantly retain Arsenazo III-Ca²⁺ complexes.

13. The method of claim 12, wherein the ratio of the amount of retained DP-Fe²⁺ complexes to that of Arsenazo III-Ca²⁺ complexes is 10:1.

14. The method of claim 12, wherein the ratio of the amount of retained DP-Fe²⁺ complexes to that of Arsenazo III-Ca²⁺ complexes is 100:1.

15. The method of claim 12, wherein the ratio of the amount of retained DP-Fe²⁺ complexes to that of Arsenazo III-Ca²⁺ complexes is 1,000:1.

16. The method of claim 12, wherein the ratio of the amount of retained DP-Fe²⁺ complexes to that of Arsenazo III-Ca²⁺ complexes is greater than 1,000:1.
